(19) United States
(12) Patent Application Publication
(10) Pub. No.: US 2024/0122452 A1
(43) Pub. Date: Apr. 18, 2024

(54) EXPANDABLE DEVICES AND SYSTEMS, AND ASSOCIATED METHODS FOR NAVIGATING TORTUOUS BODY PASSAGES

(71) Applicant: BOSTON SCIENTIFIC SCIMED, INC., Maple Grove, MN (US)

(72) Inventors: Gian Franco Loo Fuchs, San Jose (CR); Yeison Calvo, San Ramón (CR); Gonzalo Jose Saenz Villalobos, Alajuela (CR); Sean P. Fleury, Princeton, MA (US); Molly Phillips-Hungerford, Somerville, MA (US)

(73) Assignee: BOSTON SCIENTIFIC SCIMED, INC., MAPLE GROVE, MN (US)

(21) Appl. No.: 18/379,489

(22) Filed: Oct. 12, 2023

Related U.S. Application Data
(60) Provisional application No. 63/416,172, filed on Oct. 14, 2022.

(51) Int. Cl. A61B 1/00; A61B 1/005

(52) U.S. Cl. CPC A61B 1/00082; A61B 1/00085; A61B 1/0053; A61B 1/0057

(57) ABSTRACT

Devices, systems, and methods facilitating navigation through a body passage, particularly a tortuous body passage. An expandable member is mounted with respect to an element of the device or system and is shiftable from a delivery configuration to an expanded configuration. In the expanded configuration, the expandable member facilitates navigation of the device and/or system through a tortuous section of the body passage. The expandable member may include an expandable basket-like element formed of one or more elements shiftable from a generally elongated configuration when the expandable member is in the delivery configuration to a curved or bowed configuration when the expandable member is in the expanded configuration. Additionally or alternatively, the expandable member may include an inflatable balloon. The system and method may include delivering a device such as a locator element to a target site to be identified from a remote location.

### CROSS-REFERENCE TO RELATED APPLICATIONS

This application claims the benefit of priority under 35 U.S.C. § 119 to U.S. Provisional Application No. 63/416,172, filed Oct. 14, 2022, the entire disclosure of which is hereby incorporated by reference herein for all purposes.

### FIELD

The present disclosure relates generally to devices, systems, and methods used in performing procedures within a body, such as endoscopic procedures. More particularly, the present disclosure relates generally to devices, systems, and methods used to navigate to an anatomical site within a patient's body, such as a site at which a procedure is to be performed. Even more particularly, the present disclosure relates to devices, systems, and methods for facilitating navigation, delivery, and deployment of a device or system through a tortuous body passage to a target site.

### BACKGROUND

Various medical procedures require navigation of instruments, devices, tools, etc., through tortuous body passages, such as the small intestines. Medical professionals may face challenges navigating even highly flexible delivery devices, such as guidewires, through such tortuous body passages. The pushability and trackability of a guidewire through the small intestines present considerable challenges because of the tortuosity of the small intestines and variations among anatomies of patients. Moreover, a flexible guidewire may be subject to loop (e.g., loop back on itself) within tortuous body passages or certain anatomical structures. Without the ability to advance a flexible elongate element to a target site (e.g., a treatment site) to deliver a device to the target site, further procedures cannot be performed. It is with respect to these and other considerations that the present improvements may be useful.

### SUMMARY

This Summary is provided to introduce, in simplified form, a selection of concepts described in further detail below in the Detailed Description. This Summary is not intended to necessarily identify key features or essential features of the claimed subject matter, nor is it intended as an aid in determining the scope of the claimed subject matter. One of skill in the art will understand that each of the various aspects and features of the present disclosure may advantageously be used separately in some instances, or in combination with other aspects and features of the disclosure in other instances, whether or not described in this Summary. No limitation as to the scope of the claimed subject matter is intended by either the inclusion or non-inclusion of elements, components, or the like in this Summary.

In accordance with various principles of the present disclosure, a system, navigable within a patient's anatomy to a target site within the patient, includes a flexible elongate delivery device having a distal end navigable within the patient's body and a proximal end accessible for navigating the flexible elongate delivery device; a device carried by the flexible elongate delivery device along a distal end of the flexible elongate delivery device for deployment at the target site; and an expandable member mounted with respect to the flexible elongate delivery device and shiftable between a delivery configuration and an expanded configuration with a cross-sectional dimension greater than the delivery configuration. The expandable member may be mounted adjacent to the device carried by the flexible elongate delivery device.

In some embodiments, the expandable member includes one or more flexible elongated elements shiftable from a generally elongated delivery configuration to the expanded configuration. In some embodiments, the one or more flexible elongated elements are bowed or curved when in the expanded configuration. In some embodiments, the one or more flexible elongated elements are formed of a shape memory material configured to shift the expandable member into the expanded configuration upon application of force thereto and to return the expandable member to the delivery configuration upon removal of the force therefrom. In some embodiments, the flexible elongate delivery device comprises a flexible tubular element and an actuator extending through the flexible tubular element, the actuator being coupled to the expandable member to shift the expandable member between the delivery configuration and the expanded configuration upon axial translation of the actuator. In some embodiments, proximal ends of the one or more flexible elongated elements are coupled to a distal end of the flexible tubular element, and distal ends of the one or more flexible elongated elements are coupled to the actuator. In some embodiments, proximal retraction of the actuator causes the one or more flexible elongated elements to expand the expandable member into the expanded configuration.

In some embodiments, the expandable member comprises an inflatable balloon. In some embodiments, the system further includes an inflation lumen extending along the flexible elongate delivery device and in fluid communication with the inflatable balloon to deliver an inflation medium thereto to cause the inflatable balloon to shift from the delivery configuration to the expanded configuration.

In some embodiments, the expandable member is mounted adjacent the distal end of the flexible elongate delivery device, the system further comprising a locator element mounted along or adjacent the distal end of the flexible elongate delivery device.

In some embodiments, the expandable member is expandable to have a diameter at least half the inner diameter of a body passage through which the system is to be navigated.

In accordance with various principles of the present disclosure, a system, navigable within a patient's anatomy to a target site within the patient, includes an endoscope with a working channel extending therethrough; a flexible elongate delivery device having a distal end extendable distally out of the working channel of the endoscope and navigable within the patient's body and a proximal end accessible for navigating the flexible elongate delivery device; a device carried by the flexible elongate delivery device along a distal end of the flexible elongate delivery device for deployment at the target site; and an expandable member mounted with respect to the flexible elongate delivery device and shiftable between a delivery configuration and an expanded configuration with a cross-sectional dimension greater than the delivery configuration. The expandable member may be mounted adjacent to the device carried by the flexible elongate delivery device.

In some embodiments, the expandable member includes one or more flexible elongated elements shiftable from a generally elongated delivery configuration to the expanded configuration.

In some embodiments, the expandable member is an inflatable balloon.

In some embodiments, the expandable member is mounted adjacent the distal end of the flexible elongate delivery device, and the system further includes a locator element mounted along or adjacent the distal end of the flexible elongate delivery device. In some embodiments, the system further includes a locator element mounted along or adjacent the distal end of the flexible elongate delivery device.

In accordance with various principles of the present disclosure, a method of navigating a device within a patient includes advancing a flexible elongate delivery device into a body passage within the patient; and expanding an expandable member mounted adjacent a distal end of the flexible elongate delivery device from a delivery configuration to an expanded configuration when the distal end of the flexible elongate delivery device is advanced into a tortuous section of the body passage to facilitate navigation of the distal end of the flexible elongate delivery device past the tortuous section of the body passage.

In some aspects, the method further includes expanding the expandable member to have an outer diameter at least half the inner diameter of the tortuous section of the body passage to prevent the flexible elongate delivery device from bending back on itself within the tortuous section of the body passage.

In some aspects, the method further includes expanding the expandable member to expand the tortuous section of the body passage.

In some aspects, the method further includes advancing the expandable member past the tortuous section of the body passage, and returning the expandable member to the delivery configuration.

In some aspects, the method further includes delivering a locator element mounted adjacent the distal end of the flexible elongate delivery device to a target site within the patient.

These and other features and advantages of the present disclosure, will be readily apparent from the following detailed description, the scope of the claimed invention being set out in the appended claims. While the following disclosure is presented in terms of aspects or embodiments, it should be appreciated that individual aspects can be claimed separately or in combination with aspects and features of that embodiment or any other embodiment.

### BRIEF DESCRIPTION OF THE DRAWINGS

Non-limiting embodiments of the present disclosure are described by way of example with reference to the accompanying drawings, which are schematic and not intended to be drawn to scale. The accompanying drawings are provided for purposes of illustration only, and the dimensions, positions, order, and relative sizes reflected in the figures in the drawings may vary. For example, devices may be enlarged so that detail is discernable, but is intended to be scaled down in relation to, e.g., fit within a working channel of a delivery catheter or endoscope. In the figures, identical or nearly identical or equivalent elements are typically represented by the same reference characters, and similar elements are typically designated with similar reference numbers differing in increments of 100, with redundant description omitted. For purposes of clarity and simplicity, not every element is labeled in every figure, nor is every element of each embodiment shown where illustration is not necessary to allow those of ordinary skill in the art to understand the disclosure.

The detailed description will be better understood in conjunction with the accompanying drawings, wherein like reference characters represent like elements, as follows:

FIG. 1 illustrates a perspective view of an example of an embodiment of a locator system formed in accordance with various aspects of the present disclosure being navigated through a schematic representation of a gastrointestinal environment.

FIG. 2A illustrates a perspective view of an example of an embodiment of an expandable system formed in accordance with various principles of the present disclosure and in a collapsed configuration.

FIG. 2B illustrates a perspective view of an expandable system such as illustrated in FIG. 2A, but in a partially expanded configuration.

FIG. 2C illustrates a perspective view of an expandable system such as illustrated in FIG. 2A, but in an expanded configuration.

FIG. 3A illustrates another example of an embodiment of an expandable system formed in accordance with various principles of the present disclosure and in a collapsed configuration.

FIG. 3B illustrates a perspective view of an expandable system such as illustrated in FIG. 3A, but in an expanded configuration.

FIG. 3C illustrates a perspective view of an expandable system such as illustrated in FIG. 3A and FIG. 3B, as advanced beyond a tortuous body passage.

FIG. 4A illustrates another example of an embodiment of an expandable system formed in accordance with various principles of the present disclosure and in a collapsed configuration.

FIG. 4B illustrates another example of an embodiment of an expandable system such as illustrated in FIG. 4A, but in an expanded configuration.

### DETAILED DESCRIPTION

The following detailed description should be read with reference to the drawings, which depict illustrative embodiments. It is to be understood that the disclosure is not limited to the particular embodiments described, as such may vary. All apparatuses and systems and methods discussed herein are examples of apparatuses and/or systems and/or methods implemented in accordance with one or more principles of this disclosure. Each example of an embodiment is provided by way of explanation and is not the only way to implement these principles but are merely examples. Thus, references to elements or structures or features in the drawings must be appreciated as references to examples of embodiments of the disclosure, and should not be understood as limiting the disclosure to the specific elements, structures, or features illustrated. Other examples of manners of implementing the disclosed principles will occur to a person of ordinary skill in the art upon reading this disclosure. In fact, it will be apparent to those skilled in the art that various modifications and variations can be made in the present disclosure without departing from the scope or spirit of the present subject matter. For instance, features illustrated or described as part of one embodiment can be used with another embodiment to yield a still further embodiment. Thus, it is intended that the present subject matter covers such modifications and variations as come within the scope of the appended claims and their equivalents.

It will be appreciated that the present disclosure is set forth in various levels of detail in this application. In certain instances, details that are not necessary for one of ordinary skill in the art to understand the disclosure, or that render other details difficult to perceive may have been omitted. The terminology used herein is for the purpose of describing particular embodiments only, and is not intended to be limiting beyond the scope of the appended claims. Unless defined otherwise, technical terms used herein are to be understood as commonly understood by one of ordinary skill in the art to which the disclosure belongs. All of the devices and/or methods disclosed and claimed herein can be made and executed without undue experimentation in light of the present disclosure.

As used herein, “proximal” refers to the direction or location closest to the user (medical professional or clinician or technician or operator or physician, etc., such terms being used interchangeably herein without intent to limit, and including automated controller systems or otherwise), etc., such as when using a device (e.g., introducing the device into a patient, or during implantation, positioning, or delivery), and/or closest to a delivery device, and “distal” refers to the direction or location furthest from the user, such as when using the device (e.g., introducing the device into a patient, or during implantation, positioning, or delivery), and/or closest to a delivery device. “Longitudinal” means extending along the longer or larger dimension of an element. A “longitudinal axis” extends along the longitudinal extent of an element, though is not necessarily straight and does not necessarily maintain a fixed configuration if the element flexes or bends, and “axial” generally refers to along the longitudinal axis. However, it will be appreciated that reference to axial or longitudinal movement with respect to the above-described systems or elements thereof need not be strictly limited to axial and/or longitudinal movements along a longitudinal axis or central axis of the referenced elements. “Central” means at least generally bisecting a center point and/or generally equidistant from a periphery or boundary, and a “central axis” means, with respect to an opening, a line that at least generally bisects a center point of the opening, extending longitudinally along the length of the opening when the opening comprises, for example, a tubular element, a channel, a cavity, or a bore. As used herein, a “lumen” or “channel” or “bore” or “passage” is not limited to a circular cross-section. As used herein, a “free end” of an element is a terminal end at which such element does not extend beyond. It will be appreciated that terms such as at or on or adjacent or along an end may be used interchangeably herein without intent to limit unless otherwise stated, and are intended to indicate a general relative spatial relation rather than a precisely limited location. Finally, reference to “at” a location or site is intended to include at and/or about the vicinity of (e.g., along, adjacent, etc.) such location or site.

In order to perform various medical procedures noninvasively (e.g., transluminally, without open surgery), instruments, tools, devices, etc. (such terms being used interchangeably herein without intent to limit) must be navigated through tortuous body passages. For instance, various procedures may be formed transluminally through or within the intestines. One procedure performed with respect to the small intestines is a gastrojejunostomy involving creation of an anastomosis between a patient's stomach and a portion of the patient's jejunum. The anastomosis is created from within the stomach to the target site along the jejunum. In order to locate the target site along the jejunum, a locator system may be delivered through the small intestines to the target site in the jejunum. In some embodiments, the locator system includes a beacon element delivered on a flexible elongate element, such as a flexible elongate delivery device, sufficiently flexible to be navigated through the jejunum to deliver the beacon element to the target site.

In accordance with various principles of the present disclosure, an expandable element is provided on a flexible elongate delivery device to facilitate navigation of the delivery device, through an anatomical structure, to the target site. It will be appreciated that terms such as navigate, maneuver, manipulate, control, direct, advance, etc., and other grammatical forms thereof, may be used interchangeably herein without intent to limit. In some embodiments, the expandable element is an expandable basket-type element formed of one or more elements shiftable to shift the expandable member between a delivery configuration and an expanded configuration. In some embodiments, the expandable element is an expandable balloon. Optionally, the expandable member includes both an expandable basket and an expandable balloon. The expandable element may be expandable to at least half the inner diameter of the body passage through which the locator system is navigated so that the locator system cannot double back on itself. It will be appreciated that terms or descriptions such as double back, loop, bend, etc., may be used interchangeably herein, without intent to limit, to refer to configurations which interfere with advancement of the system in the body passage. The expandable element may be expandable to expand the body passage to reconfigure the body passage so that the locator system may be more readily advanced therethrough. In some embodiments, the expandable element may be used to anchor the locator system in place at the target site. In some embodiments, the flexible elongate delivery device is configured to indicate, from outside the patient's body, the distance to which its distal end has been advanced, such as to verify the distal end has reached the desired target site. For instance, markers may be provided along or on a proximal end of the flexible elongate delivery device to indicate the extent to which the flexible elongate delivery device has been advanced. If the target site is located at a particular distance within the patient's body, then sufficient advancement of the system may be determinable from outside the patient's body. In some embodiments, the flexible elongate delivery device and/or the expandable element includes markers, such as radiopaque markers, for visualizing the position of the expandable element within the patient, such as via fluoroscopy or other acceptable imaging techniques.

Various embodiments of devices, systems, and methods with expandable elements facilitating navigation through tortuous body passages will now be described with reference to examples illustrated in the accompanying drawings. Reference in this specification to “one embodiment,” “an embodiment,” “some embodiments”, “other embodiments”, etc. indicates that one or more particular features, structures, concepts, and/or characteristics in accordance with principles of the present disclosure may be included in connection with the embodiment. However, such references do not necessarily mean that all embodiments include the particular features, structures, concepts, and/or characteristics, or that an embodiment includes all features, structures, concepts, and/or characteristics. Some embodiments may include one or more such features, structures, concepts, and/or characteristics, in various combinations thereof. It should be understood that one or more of the features, structures, concepts, and/or characteristics described with reference to one embodiment can be combined with one or more of the features, structures, concepts, and/or characteristics of any of the other embodiments provided herein. That is, any of the features, structures, concepts, and/or characteristics described herein can be mixed and matched to create hybrid embodiments, and such hybrid embodiment are within the scope of the present disclosure. Moreover, references to “one embodiment,” “an embodiment,” “some embodiments”, “other embodiments”, etc. in various places in the specification are not necessarily all referring to the same embodiment, nor are separate or alternative embodiments necessarily mutually exclusive of other embodiments. It should further be understood that various features, structures, concepts, and/or characteristics of disclosed embodiments are independent of and separate from one another, and may be used or present individually or in various combinations with one another to create alternative embodiments which are considered part of the present disclosure. Therefore, the present disclosure is not limited to only the embodiments specifically described herein, as it would be too cumbersome to describe all of the numerous possible combinations and subcombinations of features, structures, concepts, and/or characteristics, and the examples of embodiments disclosed herein are not intended as limiting the broader aspects of the present disclosure. It should be appreciated that various dimensions provided herein are examples and one of ordinary skill in the art can readily determine the standard deviations and appropriate ranges of acceptable variations therefrom which are covered by the present disclosure and any claims associated therewith. The following description is of illustrative examples of embodiments only, and is not intended as limiting the broader aspects of the present disclosure.

It will be appreciated that common features are identified by common reference elements and, for the sake of brevity and convenience, and without intent to limit, the descriptions of the common features are generally not repeated. For purposes of clarity, not all components having the same reference number are numbered. Moreover, a group of similar elements may be indicated by a number and letter, and reference may be made generally to one or such elements or such elements as a group by the number alone (without including the letters associated with each similar element). It will be appreciated that, in the following description, elements or components similar among the various illustrated embodiments with reference numbers greater than 100 are generally designated with the same reference numbers increased by a multiple of 100 and redundant description is generally omitted for the sake of brevity. Moreover, certain features in one embodiment may be used across different embodiments and are not necessarily individually labeled when appearing in different embodiments.

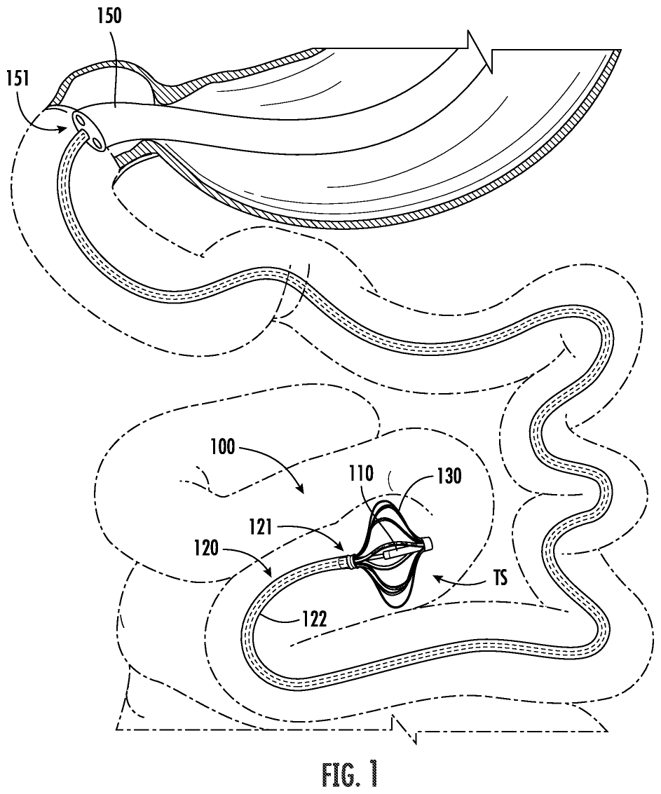

Turning now to the drawings, an example of an embodiment of a system 100 which may be used to deliver a device to a target site TS is illustrated in FIG. 1. In accordance with various principles of the present disclosure, the system 100 illustrated in FIG. 1 includes a flexible elongate delivery device 120 carrying a device 110. For the sake of convenience, and without intent to limit, reference is made to a locator element 110 rather than simply a device. However, it will be appreciated that although the illustrated example of an embodiment of a system 100 is configured to deliver a locator element 110, other devices, as well as other configurations, uses, etc., of a system 100 formed in accordance with various principles of the present disclosure are within the scope and spirit of the present disclosure. Moreover, although the system 100 is illustrated in a gastrointestinal environment, principles of the present disclosure are applicable for use in other anatomical sites as well.

As further illustrated in FIG. 1, in accordance with various principles of the present disclosure, an expandable member 130 is positioned adjacent the distal end 121 of the flexible elongate delivery device 120. The expandable member 130 may be carried adjacent the distal end 121 of the flexible elongate delivery device 120, distal to, proximal to, or coextensive with the locator element 110, as described in further detail below. The expandable member 130 is selectively expandable from a contracted or collapsed delivery configuration to an expanded configuration having a cross-sectional dimension greater than that of the cross-sectional dimension when in the delivery configuration. In the delivery configuration, the expandable member 130 is capable of being delivered transluminally (e.g., with an endoscope or other tubular delivery element, such as a delivery catheter or sheath, and/or through body passages) to an anatomical site. In certain passages, such as tortuous body passages, expansion of the expandable member 130 into the expanded configuration facilitates advancement of the system 100 therethrough. For instance, expansion of the expandable member 130 to a diameter at least half the inner diameter of the body passage through which the system 100 is being advanced may prevent looping of the expandable member 130 and thereby facilitate distal advancement of the system 100 through the body passage. Additionally or alternatively, expansion of the expandable member 130 may straighten or otherwise adjust the configuration of a tortuous body passage to facilitate advancement therethrough. Once the system 100 is capable of being advanced further (such as, but not necessarily, while still in the expanded configuration), as desired, the system 100 may be advanced further in the delivery configuration. In some embodiments, the system 100 may be returned to the expanded configuration once at the target site TS for various purposes as will be described below with reference to the illustrated examples of embodiments of systems formed in accordance with various principles of the present disclosure.

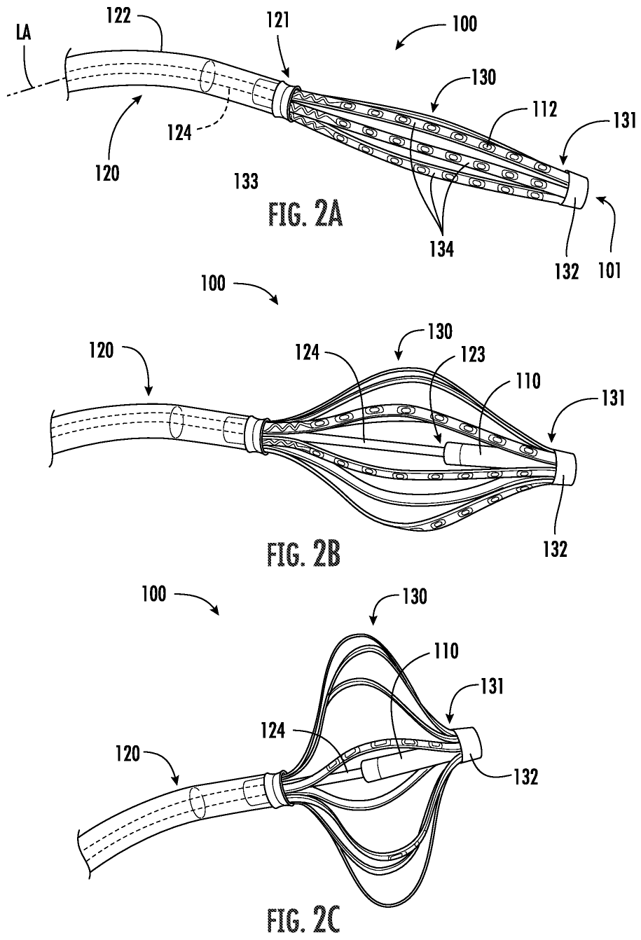

The example of an embodiment of a system 100 illustrated in FIG. 1 is illustrated in further detail in FIG. 2A, FIG. 2B, and FIG. 2C, showing various configurations of the expandable member 130. The expandable member 130 is illustrated in FIG. 2A in a collapsed configuration extending generally along the distal end 101 of the system 100. As may be appreciated with reference to FIG. 2B and FIG. 2C, the flexible elongate delivery device 120 of the illustrated example of an embodiment of a system 100 includes a flexible tubular element 122 and an actuator 124. The actuator 124 may be a core wire, pull wire, guidewire, or other flexible elongate element capable of axial translation within the flexible tubular element 122. As may be appreciated with reference to FIG. 2A, FIG. 2B, and FIG. 2C, the expandable member 130 extends distally from a distal end 121 of the flexible tubular element 122 of the flexible elongate delivery device 120. And, as may be appreciated with reference to FIG. 2B and FIG. 2C, the actuator 124 extends through the flexible tubular element 122 and along (e.g., through) the expandable member 130 to a distal end 131 of the expandable member 130. The distal end 123 of the actuator 124 is coupled to the distal end 131 of the expandable member 130. In some embodiments, an end cap 132 (e.g., a cylindrical collar or ring open at both ends or just at one end) is provided at (mounted on, coupled to, etc.) the distal end 131 of the expandable member 130, such as to receive the distal end 131 of the expandable member 130. In some embodiments, the distal end 123 of the actuator 124 is coupled to the end cap 132 in addition to or instead of the distal end 131 of the expandable member 130. The end cap 132 may be coupled to the distal end 131 of the expandable member 130 and/or to the actuator 124, and the proximal end 133 of the expandable member 130 may be coupled to the distal end 121 of the flexible tubular element 122 in any of a variety of manners known to those of ordinary skill in the art, such as bonding (e.g., adhesive bonding, welding, brazing, soldering, etc.), mechanical interference (e.g., crimping, friction fit, etc.), mechanical coupling (e.g., riveting, threaded engagement, etc.) etc. The proximal end of the flexible elongate delivery device 120, and, more particularly, the proximal ends of the flexible tubular element 122 and the actuator 124 (not shown, but the configurations of which are readily understandable by those of ordinary skill in the art) extend proximally to a position allowing control thereof by a medical professional. A control handle, such as any known to those of ordinary skill in the art, may be provided, or the proximal ends may be directly grasped and maneuvered by the medical professional. In some embodiments, the control handle includes an actuator or trigger or other mechanism known to those of ordinary skill in the art configured to allow the medical professional to actuate the actuator 124 to control shifting of the configuration of the expandable member 130.

In accordance with various principles of the present disclosure, proximal retraction of the actuator 124 of the example of an embodiment of a system 100 illustrated in FIGS. 2A, 2B, and 2C brings the distal end 131 of the illustrated example of an embodiment of an expandable member 130 closer to the proximal end 133 of the expandable member 130 and causes expansion of the expandable member 130. Such actuation may be achieved in a variety of manners known to those of ordinary skill in the art. For instance, the illustrated example of an embodiment of an expandable member 130 is formed of one or more flexible elongated elements 134, such as wires, ribbons, bands, filaments, etc. The flexible elongated elements 134 are formed of a material allowing the flexible elongated elements 134 to bend or bow or curve outwardly to expand the cross-sectional dimension (transverse to the longitudinal axis LA of the system 100) of the expandable member 130, shifting the expandable member 130 from the generally elongated delivery configuration illustrated in FIG. 2A, to a transition configuration as illustrated FIG. 2B, to an expanded configuration as illustrated in FIG. 2C, such as with the assistance of the actuator 124. As such, the system 100 may be advanced through a body passage when in the delivery configuration illustrated in FIG. 2A. When the system 100 approaches or is advanced into a particularly tortuous section of the body passage (e.g., a bend or kink in the small intestines), the expandable member 130 may be expanded to facilitate advancement of the system 100 through the body passage. For instance, if the expandable member 130 is expanded to at least half the inner diameter of the body passage, the system 100 cannot double back over itself because it would be too wide to fit within the body passage. Additionally or alternatively, expansion of the expandable member 130 may cause the body passage to be expanded and reconfigured (e.g., straightened, elongated, etc.) to facilitate passage of the expandable member 130 and the system 100 therethrough. Additionally or alternatively, expansion of the expandable member 130 helps to expand the body passage in which the expandable member 130 is positioned and/or to move tissue out of the way to facilitate easier delivery. For instance, the expandable member 130, when positioned in a patient's intestines, may open the intestines which otherwise would be collapsed (because the patient typically is not allowed to eat prior to the procedure being performed and therefore there is no food in the intestines to hold open the walls of the intestines). Once the system 100 is able to navigate past the tortuous body passage, the expandable member 130 may be returned to its delivery configuration, such as illustrated in FIG. 2C, or optionally to a configuration between the delivery configuration and the expanded configuration. The system 100 may then be advanced to the target site TS and the expandable member 130 may be delivered to the target site TS, such as illustrated in FIG. 1.

As noted above, the expandable member 130 may be expanded to facilitate navigation through a particular anatomical structure, such as a tortuous body passage, and then returned to an unexpanded delivery configuration once past such particular anatomical structure. To facilitate return of the expandable member 130 to a delivery configuration, one or more elements of the expandable member 130 may be formed of shape memory material returning the expandable member 130 to its delivery configuration. The shape memory material may be selected from one or more, preferably biocompatible, metals (e.g., stainless steel, nitinol, Elgiloy®, etc.), polymers, or combinations thereof. In such embodiment, the expandable member 130 is shifted into the expanded configuration upon application of force thereto, whereas the expandable member 130 returns to the delivery configuration upon removal of such force.

In the example of an embodiment of a system 100 illustrated in FIG. 1, FIG. 2A, FIG. 2B, and FIG. 2C, the flexible elongate delivery device 120 delivers a device 110 to a target site TS. As noted above, and as may be appreciated with reference to FIG. 2B and FIG. 2C, the device 110 may include a locator element 110. Although the locator element 110 is illustrated positioned generally along the expandable member 130, it will be appreciated that other relative positions of these elements are within the scope and spirit of the present disclosure. For instance, the locator element 110 may be proximal or distal to the expandable member 130 without detracting from aspects or structures or functions of the system 100. In the example of an embodiment illustrated in FIG. 2B and FIG. 2C, the locator element 110 is mounted on the actuator 124 and generally within the expandable member 130. It will be appreciated that other configurations and positions of a device 110 are within the scope and spirit of the present disclosure. For instance, the material of the expandable member 130 and/or a marker (e.g., a radiopaque marker) may be imagable by various imaging systems and may thus function as a device delivered by the system 100 for locating a target site TS within a body passage from a remote, different location (outside or within the patient). For instance, the expandable member 130 may be formed of a material capable of being imaged, such as a metal. As such, the expandable member 130 may also function as a locator element 110 which may be used to identify the target site TS at which the expandable member 130 has been deployed. Optionally, one or more additional locator elements 112, such as radiopaque markers, may be provided on the expandable member 130, such as on one or more or each of the flexible elongated elements 134. For instance, a plurality of additional locator elements 112 may be provided along the longitudinal extent of at least one of the flexible elongated elements 134, such as illustrated in FIG. 2A and FIG. 2B.

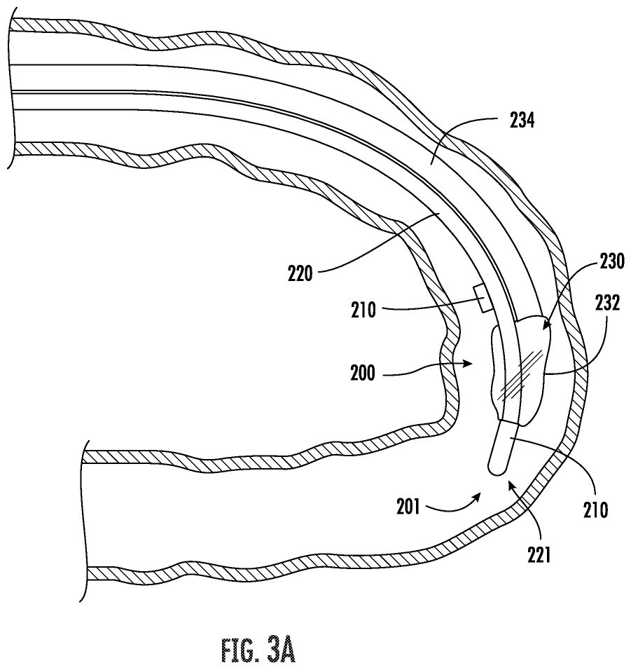

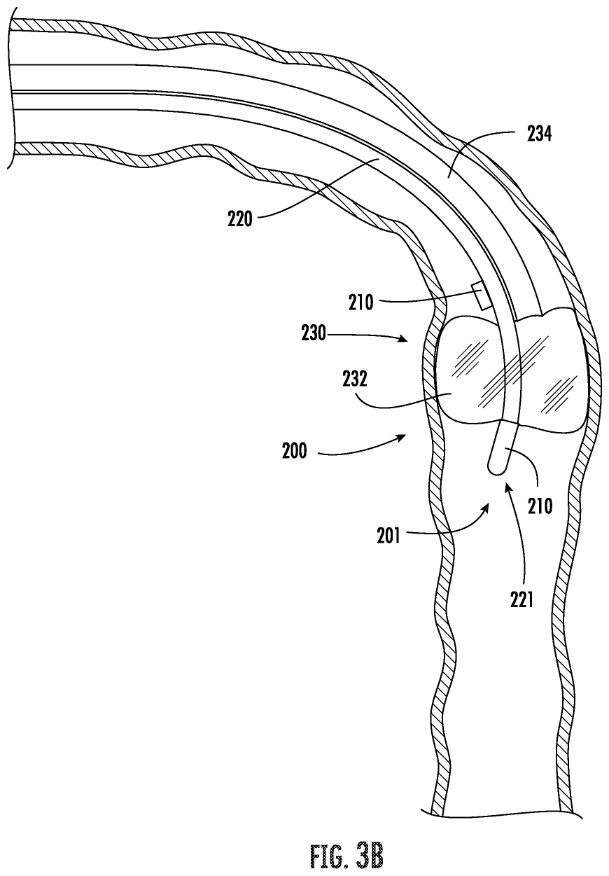

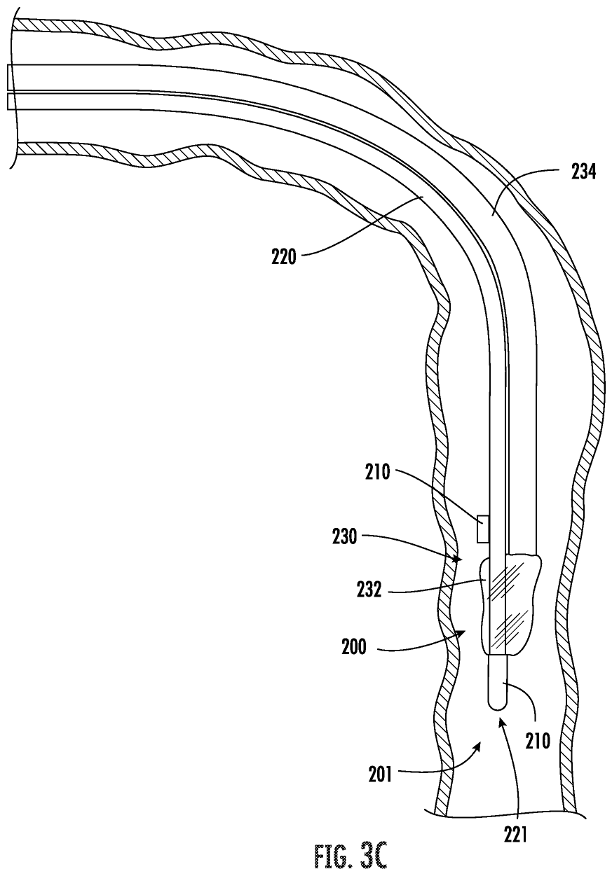

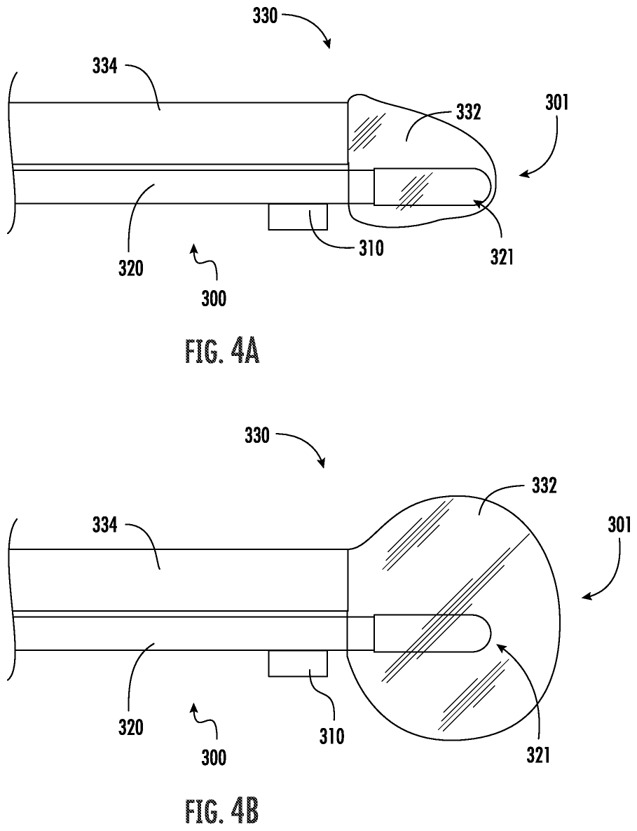

An expandable member of a system formed in accordance with various principles of the present disclosure may be formed in any of a variety of manners other than the example of an embodiment illustrated in FIG. 2A, FIG. 2B, and FIG. 2C. For instance, in the example of an embodiment of a system 200 illustrated in FIG. 3A, FIG. 3B, and FIG. 3C, and in the example of an embodiment of a system 300 illustrated in FIG. 4A and FIG. 4B, a flexible elongate delivery device 220, 320 is navigated with the assistance of an expandable member 230, 330 including an expandable balloon element 232, 332. The expandable balloon element 232 may be positioned proximal to the distal end 201 of the system 200 (e.g., proximal to the distal end 221 of the flexible elongate delivery device 220), such as illustrated in FIG. 3A, FIG. 3B, and FIG. 3C. Alternatively, the expandable member 330 may be positioned over the distal end 301 of the system 300 (e.g., over the distal end 321 of the flexible elongate delivery device 320), such as illustrated in FIG. 4A and FIG. 4B. An inflation lumen 234, 334 may be fluidly coupled/in fluid communication with the expandable member 230, 330 to facilitate expansion of the expandable member 230, 330. For instance, the inflation lumen 234, 334 may extend from a respective expandable member 230, 330 to an inflation source at a proximal end thereof to deliver an inflation medium to the expandable member 230, 330 such as in manner known to those of ordinary skill in the art. The inflation lumen 234, 334 may be positioned with respect to the flexible elongate delivery device 220, 320 in any of a variety of manners, such as mounted thereon (e.g., with any of a variety of mounting elements such as clips), or otherwise secured thereto (e.g., with adhesive, or with a sleeve, such as formed from a shrink wrap element) extending therearound).

The respective expandable members 230, 330 of the systems 200, 300 illustrated, respectively, in FIG. 3A, FIG. 3B, and FIG. 3C and FIG. 4A and FIG. 4B may facilitate navigation of the systems 200, 300 in a manner similar to that described above with respect to the system 100 illustrated in FIG. 1, FIG. 2A, FIG. 2B, and FIG. 2C. Various configurations and stages of use of an expandable member 230 which may facilitate navigation of the system 200 are illustrated in FIG. 3A, FIG. 3B, and FIG. 3C. Specifically, the expandable member 230 of the system 200 is illustrated in a delivery configuration in FIG. 3A, extending into a tortuous body passage. As illustrated in FIG. 3B, the expandable member 230 is expandable to facilitate navigation of the system 200 through the tortuous body passage. Once the system 200 is able to navigate past the tortuous body passage, the expandable member 230 may be returned to its delivery configuration, such as illustrated in FIG. 3C, or optionally to a configuration between the delivery configuration and the expanded configuration. Similarly, FIG. 4A illustrates an example of an embodiment of a system 300 with its expandable member 330 in a delivery configuration, and FIG. 4B illustrates the expandable member 330 in an expanded configuration which may facilitate navigation of the system 300. Such configurations of the system 300 may be navigated through a patient and through a tortuous body passage of the passage in a manner as described with respect to FIG. 3A, FIG. 3B, and FIG. 3C, reference being made thereto for the sake of brevity.

As in the example of an embodiment of a system 100 illustrated in FIG. 1, FIG. 2A, FIG. 2B, and FIG. 2C, the flexible elongate delivery devices 220, 320 of the systems 200, 300 illustrated in FIG. 3A, FIG. 3B, and FIG. 3C and FIG. 4A and FIG. 4B, respectively, may deliver a device 210, 310 to a target site TS. The device 210, 310 may be a locator element 210, 310, such as an LED or marker visible by any of a variety of imaging techniques (such as described above with respect to the locator element 110), or any of another variety of devices known to those of ordinary skill in the art. The locator element 210, 310 may be positioned proximal to the expandable member 230, 330, as illustrated, or distal or along or on the expandable member 230, 330 without detracting from aspects or structures or functions of the system 200, 300. Optionally, a sheath or sleeve or similar element such as known to those of ordinary skill in the art is provided over elements of the system 200, 300 (e.g., over one or more of the expandable member 230, 330, the inflation lumen 234, 334, the device 210, 310, the flexible elongate delivery device 220, 320) to hold or maintain such elements in the desired relative positions with respect to one another.

Any of the systems 100, 200, 300 such as illustrated in FIG. 1, FIG. 2A, FIG. 2B, FIG. 2C, or in FIG. 3A, FIG. 3B, FIG. 3C, or in FIG. 4A, or FIG. 4B may be delivered via an endoscope 150, such as illustrated in FIG. 1. The endoscope 150 may not be able to extend the full distance to the target site TS, and the system 100, 200, 300 is thus extendable beyond the distal end 151 of the endoscope 150, such as illustrated in FIG. 1. Once the system 100, 200, 300 has been navigated through the patient, such as described above, and the distal end 101, 201, 301 thereof has reached a target site, a device 110, 210, 310 may be delivered/deployed by the system 100, 200, 300, such as described above, and the endoscope 150 withdrawn. In some embodiments, the device 110, 210, 310 may be left in place at the target site TS, such as with the expandable member 130, 230, 330 in an expanded configuration to hold the device 110, 210, 310 in place, withdrawing at least a portion of the system 100, 200, 300 (e.g., at least a portion of the flexible elongate delivery device 120, 220, 320) from the target site TS.

It will be appreciated that other than the above-described differences, the systems and device described above can be arranged and operate in substantially the same or similar manners. Accordingly, for the sake of brevity and convenience, and without intent to limit, common elements with common functions have been indicated with the same reference characters differing in value by 100, reference being made to the description of similar elements and operations.

It will be appreciated that devices, systems, and methods described herein may be used with procedures to be performed in various locations within a patient's anatomy, such as the abdominal cavity, digestive system, urinary tract, reproductive tract, respiratory system, cardiovascular system, circulatory system, and the like. Therefore, although embodiments of the present disclosure have been described with specific reference to the GI system, it should be appreciated that devices and systems as described herein are not so limited. For instance, as used in the GI system, devices, systems, and methods disclosed herein may be used in performing a gastrojejunostomy by identifying a location at which an anastomosis with the patient's stomach may be created. However, it will be appreciated that system, devices, systems, and methods disclosed herein may be used with a variety of other medical procedures, such as performed in ductal, luminal, vascular, or body lumen anatomies, including, for example, interventional radiology procedures, balloon angioplasty/angiography procedures, thrombolysis procedures, urological or gynecological procedures, and the like. The medical devices herein may include a variety of medical devices for navigating body lumens, including, for example, catheters, ureteroscopes, bronchoscopes, colonoscopes, arthroscopes, cystoscopes, hysteroscopes, and the like. The disclosed medical devices and systems may also be inserted via different access points and approaches, e.g., percutaneously, endoscopically, laparoscopically, or combinations thereof.

It is to be understood by one of ordinary skill in the art that the present discussion is a description of illustrative examples of embodiments only, and is not intended as limiting the broader aspects of the present disclosure. All apparatuses and methods discussed herein are examples of apparatuses and/or methods implemented in accordance with one or more principles of this disclosure. These examples are not the only way to implement these principles but are merely examples, not intended as limiting the broader aspects of the present disclosure. Thus, references to elements or structures or features in the drawings must be appreciated as references to examples of embodiments of the disclosure, and should not be understood as limiting the disclosure to the specific elements, structures, or features illustrated. Other examples of manners of implementing the disclosed principles will occur to a person of ordinary skill in the art upon reading this disclosure. It should be apparent to those of ordinary skill in the art that variations can be applied to the disclosed devices, systems, and/or methods, and/or to the sequence of steps of the method described herein without departing from the concept, spirit, and scope of the disclosure. It will be appreciated that various features described with respect to one embodiment typically may be applied to another embodiment, whether or not explicitly indicated. The various features hereinafter described may be used singly or in any combination thereof. Therefore, the present invention is not limited to only the embodiments specifically described herein, and all substitutes and modifications apparent to those skilled in the art are deemed to be within the spirit, scope, and concept of the disclosure as defined by the appended claims. Various further benefits of the various aspects, features, components, and structures of a delivery device or system and associated methods of use, such as described above, in addition to those discussed above, may be appreciated by those of ordinary skill in the art.

The foregoing discussion has broad application and has been presented for purposes of illustration and description and is not intended to limit the disclosure to the form or forms disclosed herein. It will be understood that various additions, modifications, and substitutions may be made to embodiments disclosed herein without departing from the concept, spirit, and scope of the present disclosure. In particular, it will be clear to those skilled in the art that principles of the present disclosure may be embodied in other forms, structures, arrangements, proportions, and with other elements, materials, and components, without departing from the concept, spirit, or scope, or characteristics thereof. For example, various features of the disclosure are grouped together in one or more aspects, embodiments, or configurations for the purpose of streamlining the disclosure. However, it should be understood that various features of the certain aspects, embodiments, or configurations of the disclosure may be combined in alternate aspects, embodiments, or configurations. While the disclosure is presented in terms of embodiments, it should be appreciated that the various separate features of the present subject matter need not all be present in order to achieve at least some of the desired characteristics and/or benefits of the present subject matter or such individual features. One skilled in the art will appreciate that the disclosure may be used with many modifications or modifications of structure, arrangement, proportions, materials, components, and otherwise, used in the practice of the disclosure, which are particularly adapted to specific environments and operative requirements without departing from the principles or spirit or scope of the present disclosure. For example, elements shown as integrally formed may be constructed of multiple parts or elements shown as multiple parts may be integrally formed, the operation of elements may be reversed or otherwise varied, the size or dimensions of the elements may be varied. Similarly, while operations or actions or procedures are described in a particular order, this should not be understood as requiring such particular order, or that all operations or actions or procedures are to be performed, to achieve desirable results. Additionally, other implementations are within the scope of the following claims. In some cases, the actions recited in the claims can be performed in a different order and still achieve desirable results. The presently disclosed embodiments are therefore to be considered in all respects as illustrative and not restrictive, the scope of the claimed subject matter being indicated by the appended claims, and not limited to the foregoing description or particular embodiments or arrangements described or illustrated herein. In view of the foregoing, individual features of any embodiment may be used and can be claimed separately or in combination with features of that embodiment or any other embodiment, the scope of the subject matter being indicated by the appended claims, and not limited to the foregoing description.

In the foregoing description and the following claims, the following will be appreciated. The phrases “at least one”, “one or more”, and “and/or”, as used herein, are open-ended expressions that are both conjunctive and disjunctive in operation. The terms “a”, “an”, “the”, “first”, “second”, etc., do not preclude a plurality. For example, the term “a” or “an” entity, as used herein, refers to one or more of that entity. As such, the terms “a” (or “an”), “one or more” and “at least one” can be used interchangeably herein. As used in this specification and the appended claims, the term “or” is generally employed in its sense including “and/or” unless the content clearly dictates otherwise. As used herein, the conjunction “and” includes each of the structures, components, features, or the like, which are so conjoined, unless the context clearly indicates otherwise, and the conjunction “or” includes one or the others of the structures, components, features, or the like, which are so conjoined, singly and in any combination and number, unless the context clearly indicates otherwise. All directional references (e.g., proximal, distal, upper, lower, upward, downward, left, right, lateral, longitudinal, front, back, top, bottom, above, below, vertical, horizontal, radial, axial, clockwise, counterclockwise, and/or the like) are only used for identification purposes to aid the reader's understanding of the present disclosure, and/or serve to distinguish regions of the associated elements from one another, and do not limit the associated element, particularly as to the position, orientation, or use of this disclosure. Connection references (e.g., attached, coupled, connected, engaged, and joined) are to be construed broadly and may include intermediate members between a collection of elements and relative movement between elements unless otherwise indicated. As such, connection references do not necessarily infer that two elements are directly connected and in fixed relation to each other. Identification references (e.g., primary, secondary, first, second, third, fourth, etc.) are not intended to connote importance or priority, but are used to distinguish one feature from another.

The following claims are hereby incorporated into this Detailed Description by this reference, with each claim standing on its own as a separate embodiment of the present disclosure. In the claims, the terms “comprises”, “comprising”, “includes”, and “including” do not exclude the presence of other elements, components, features, groups, regions, integers, steps, operations, etc. Additionally, although individual features may be included in different claims, these may possibly advantageously be combined, and the inclusion in different claims does not imply that a combination of features is not feasible and/or advantageous. In addition, singular references do not exclude a plurality. Reference signs in the claims are provided merely as a clarifying example and shall not be construed as limiting the scope of the claims in any way.

What is claimed is:

1. A system navigable within a patient's anatomy to a target site within the patient, said system comprising:
a flexible elongate delivery device having a distal end navigable within the patient's anatomy and a proximal end accessible for navigating said flexible elongate delivery device;
a device carried by said flexible elongate delivery device along a distal end of said flexible elongate delivery device for deployment at the target site; and
an expandable member mounted adjacent to said device and shiftable between a delivery configuration and an expanded configuration with a cross-sectional dimension greater than the delivery configuration.

2. The system of claim 1, wherein said expandable member comprises one or more flexible elongated elements shiftable from a generally elongated delivery configuration to the expanded configuration.

3. The system of claim 2, wherein said one or more flexible elongated elements are bowed or curved when in the expanded configuration.

4. The system of claim 2, wherein said one or more flexible elongated elements are formed of a shape memory material configured to shift said expandable member into the expanded configuration upon application of force thereto and to return said expandable member to the delivery configuration upon removal of the force therefrom.

5. The system of claim 2, wherein said flexible elongate delivery device comprises a flexible tubular element and an actuator extending through said flexible tubular element, said actuator being coupled to said expandable member to shift said expandable member between the delivery configuration and the expanded configuration upon axial translation of said actuator.

6. The system of claim 5, wherein proximal ends of said one or more flexible elongated elements are coupled to a distal end of said flexible tubular element, and distal ends of said one or more flexible elongated elements are coupled to said actuator.

7. The system of claim 6, wherein proximal retraction of said actuator causes said one or more flexible elongated elements to expand said expandable member into the expanded configuration.

8. The system of claim 1, wherein said expandable member comprises an inflatable balloon.

9. The system of claim 8, further comprising an inflation lumen extending along said flexible elongate delivery device and in fluid communication with said inflatable balloon to deliver an inflation medium thereto to cause said inflatable balloon to shift from the delivery configuration to the expanded configuration.

10. The system of claim 1, wherein said expandable member is mounted adjacent the distal end of said flexible elongate delivery device, said system further comprising a locator element mounted along or adjacent the distal end of said flexible elongate delivery device.

11. The system of claim 1, wherein said expandable member is expandable to have a diameter at least half an inner diameter of a body passage through which said system is to be navigated.

12. A system navigable within a patient's anatomy to a target site within the patient, said system comprising:
an endoscope with a working channel extending therethrough;
a flexible elongate delivery device having a distal end extendable distally out of the working channel of said endoscope and navigable within the patient's anatomy and a proximal end accessible for navigating said flexible elongate delivery device;
a device carried by said flexible elongate delivery device along a distal end of said flexible elongate delivery device for deployment at the target site; and
an expandable member mounted adjacent to said device and shiftable between a delivery configuration and an expanded configuration with a cross-sectional dimension greater than the delivery configuration.

13. The system of claim 12, wherein said expandable member comprises one or more flexible elongated elements shiftable from a generally elongated delivery configuration to the expanded configuration.

14. The system of claim 12, wherein said expandable member comprises an inflatable balloon.

15. The system of claim 12, wherein said expandable member is mounted adjacent the distal end of said flexible elongate delivery device, said system further comprising a locator element mounted along or adjacent the distal end of said flexible elongate delivery device, said system further comprising a locator element mounted along or adjacent the distal end of said flexible elongate delivery device.

16. A method of navigating a device within a patient, said method comprising:
advancing a flexible elongate delivery device into a body passage within the patient; and
expanding an expandable member mounted adjacent a distal end of the flexible elongate delivery device from a delivery configuration to an expanded configuration when the distal end of the flexible elongate delivery device is advanced into a tortuous section of the body passage to facilitate navigation of the distal end of the flexible elongate delivery device past the tortuous section of the body passage.

17. The method of claim 16, further comprising expanding the expandable member to have an outer diameter at least half the inner diameter of the tortuous section of the body passage to prevent the flexible elongate delivery device from bending back on itself within the tortuous section of the body passage.

18. The method of claim 16, further comprising expanding the expandable member to expand the tortuous section of the body passage.

19. The method of claim 16, further comprising advancing the expandable member past the tortuous section of the body passage, and returning the expandable member to the delivery configuration.

20. The method of claim 16, further comprising delivering a locator element mounted adjacent the distal end of the flexible elongate delivery device to a target site within the patient.
